(19) United States
(12) Patent Application Publication
(10) Pub. No.: US 2022/0291207 A1
(43) Pub. Date: Sep. 15, 2022

(54) HIGH-BRIGHTNESS FLUOROPHORES FOR QUANTIFICATION AND PHENOTYPING OF EXTRACELLULAR VESICLES

(71) Applicant: Michigan Technological University, Houghton, MI (US)

(72) Inventors: Yoke Khin Yap, Houghton, MI (US); Dongyan Zhang, Houghton, MI (US); Nazmiye Yapici, South Lyon, MI (US)

(21) Appl. No.: 17/637,018

(22) PCT Filed: Aug. 21, 2020
(86) PCT No.: PCT/US2020/047378

§ 371 Date: Feb. 21, 2022

Related U.S. Application Data
(63) Continuation-in-part of application No. PCT/US2020/035568, filed on Jun. 1, 2020.
Application No. PCT/US2020/035568 is a continuation-in-part of application No. PCT/US2020/035574, filed on Jun. 1, 2020.
(60) Provisional application No. 62/889,691, filed on Aug. 21, 2019.

(51) Int. Cl. G01N 33/533; G01N 33/50; G01N 33/543

(52) U.S. Cl. CPC G01N 33/533; G01N 33/5076; G01N 33/54346; B82Y 15/00

(57) ABSTRACT

A compound includes a nanomaterial carrier, a first linker having a first end connected to the nanomaterial carrier, a second linker having a second end connected to the nanomaterial carrier, a fluorescent entity connected to a second end of the first linker, and a biomolecule connected to a second end of the second linker. The biomolecule is configured to connect to a cluster of differentiation (CD) of an extracellular vesicle (EV). A method is also disclosed.

### CROSS-REFERENCE TO RELATED APPLICATIONS

This application claims priority to U.S. Provisional Application Ser. No. 62/889,691 filed Aug. 21, 2019, International Application No. PCT/US2020/035568, filed Jun. 1, 2020, and International Application No. PCT/US2020/035574, filed Jun. 1, 2020. U.S. Provisional Application Ser. No. 62/889,691, International Application No. PCT/US2020/035568, International Application No. PCT/US2020/035574 are hereby incorporated by reference herein in their entireties.

### STATEMENT OF GOVERNMENT SUPPORT

The inventions described herein were made with government support under Grant #1261910, Grant #1521057 and Grant # 1738466 awarded by the National Science Foundation. The Government has certain rights in this invention.

### BACKGROUND

Fluorophores are compounds with fluorescent properties that have biomedical applications. For example, fluorophores can be used as tracers or dyes for staining certain molecules or structures. More particularly, fluorophores can be used to stain tissues, cells, or biological materials in a variety of analytical methods, such as fluorescent imaging and spectroscopy.

Extracellular vesicles (EVs) are biological particles encapsulated with a phospholipid bilayer. EVs have diameter around 20 nanometers (nm) to a few microns but mostly are smaller than around 350 nm. Detection of EVs can be helpful in certain clinical applications such as early disease detection and treatment by diagnostic biomarkers and therapeutics. However, due to their small diameter, EVs only have a few biological markers. Therefore, it is difficult to accurately count and to phenotype small EVs. High-resolution imaging flow cytometry is the most promising method for counting EVs by laser light scattering and phenotyping them by fluorescent signals of fluorophores that tagged on them. However, it is still challenging to accurately quantify EVs by light scattering due to their small size, and low index of refraction (n˜1.3-1.4), swarming and to phenotype them due to the weak fluorescence signal from the small number of fluorophores on each EVs. These issues are hindering the validation of EVs use as diagnostic biomarkers and therapeutics.

### SUMMARY

A compound, according to an exemplary embodiment of this disclosure, among other possible things includes a nanomaterial carrier, a first linker having a first end connected to the nanomaterial carrier, a second linker having a second end connected to the nanomaterial carrier, a fluorescent entity connected to a second end of the first linker, and a biomolecule connected to a second end of the second linker. The biomolecule is configured to connect to a cluster of differentiation (CD) of an extracellular vesicle (EV).

In a further example of the foregoing, the nanomaterial carrier is a boron nitride nanotube (BNNT) or carbon nanotube (CNT).

In a further example of any of the foregoing, the nanomaterial is a nanodot.

In a further example of any of the foregoing, the first end of at least one of the first and second linkers is covalently bonded to the nanomaterial carrier.

In a further example of any of the foregoing, the first end of at least one of the first and second linkers includes a functional group, and the functional group covalently bonds the linker to the nanomaterial carrier.

In a further example of any of the foregoing, the second end of at least one of the first second linkers ins covalently bonded to the fluorescent entity or the biomolecule via a functional group.

In a further example of any of the foregoing, the first and of at least one of the first and second linkers is non-covalently bonded to the nanomaterial carrier.

In a further example of any of the foregoing, at least one of the first and second linkers is amphiphilic, and includes a hydrophobic region and a hydrophilic region. The hydrophobic region is non-covalently bonded to the nanomaterial carrier.

In a further example of any of the foregoing, the linkers has a molecular weight between about 1000 and 10000 Da.

In a further example of any of the foregoing, the nanomaterial carrier is a boron nitride nanotube.

In a further example of any of the foregoing, at least one of the first and second linkers is DSPE-PEGn (1,2-distearoyl-sn-glycero-3-phosphoethanolamine-N-[(polyethylene glycol)n]), where n is a number of polyethylene glycol (PEG) molecules in a PEG chain.

A method according to an exemplary embodiment of this disclosure, among other possible things includes linking at least one fluorescent entity and at least one biomolecule to a nanomaterial carrier. The biomolecule is configured to connect a cluster of differentiation (CD) of an extracellular vesicle (EV) to form a compound. The method also includes applying the compound to an EV such that the compound connects to the EV via the biomolecule to form a marked EV, and detecting at least one of light scattering and fluorescence of the marked EV.

In a further example of the foregoing, the carrier is a boron nitride nanotube (BNNT) carrier, a carbon nanotube (CNT) carrier, or a nanodot.

In a further example of any of the foregoing, the linking of at least one of the fluorescent entity and the biomolecule is via a linker. The linking of the linker to the nanomaterial carrier is via a covalent bond.

In a further example of any of the foregoing, a first end of the linker includes a first functional group and a second end of the linker includes a second functional group. The first functional group covalently bonds to the nanomaterial carrier and the second functional group covalently bonds to the fluorescent entity.

In a further example of any of the foregoing, the linking of at least one of the fluorescent entity and the biomolecule is via a linker. The linking of the linker to the nanomaterial carrier is via a non-covalent bond.

In a further example of any of the foregoing, the linker is amphiphilic, and includes a hydrophobic region and a hydrophilic region. The hydrophobic region is non-covalently bonded to the nanomaterial carrier.

In a further example of any of the foregoing, the linker has a molecular weight between about 1000 and 10000 Da.

In a further example of any of the foregoing, the nanomaterial carrier is a boron nitride nanotube.

In a further example of any of the foregoing, the linker is DSPE-PEGn (1,2-distearoyl-sn-glycero-3-phosphoethanolamine-N-[(polyethylene glycol)n]), where n is a number of polyethylene glycol (PEG) molecules in a PEG chain.

### BRIEF DESCRIPTION OF THE DRAWINGS

FIG. 1A schematically shows high-brightness fluorophore structures.

FIG. 1B schematically shows dye-linker structures.

FIG. 1C schematically shows antibody-linker structures.

FIG. 2A shows light scattering signals from a PBS buffer solution including EVs.

FIG. 2B shows fluorescence signals from the PBS buffer solution of FIG. 2A.

FIG. 3A shows light scattering signals from short BNNTs.

FIG. 3B shows light scattering signals from short BNNTs at 4× BNNT concentration.

FIG. 3C shows the length distribution of the short BNNTs.

FIG. 4A shows light scattering signals from short BNNTs labeled with dye-linkers.

FIG. 4B shows fluorescence signals from short BNNTs labeled with dye-linkers.

FIG. 5A shows light scattering signals from long BNNTs.

FIG. 5B shows the length distribution of the long BNNTs.

### DETAILED DESCRIPTION

Very generally, high-brightness fluorophores contain a carrier element, a fluorescent element, and a linker linking the carrier element to the fluorescent element. For biomedical applications, each of the carrier element, the linker, and the fluorescent element must be biocompatible (though the requirements for biocompatibility will vary with the particular application).

One example carrier element is a nanomaterial, such as carbon nanotubes (CNT) and boron nitride nanotubes (BNNTs), both of which are recognized as biologically compatible nanomaterials for biomedical applications such as cellular drug delivery and spectroscopy applications. However, it was previously shown that fluorescent elements linked to nanotubes exhibit quenching, or a reduction in the brightness of the fluorescence.

It has been discovered that certain fluorophores having nanomaterial carriers not only do not exhibit the quenching effect, but also that exhibit brightness several orders of magnitude higher than other known fluorophores, as has been described in U.S. patent application Ser. No. 15/953,200, filed Apr. 13, 2018, and published as U.S. Patent Pub. No. 2018/0296705; International Application No. PCT/US2020/035568, filed Jun. 1, 2020; and International Application No. PCT/US2020/035574. U.S. patent application Ser. No. 15/953,200, International Application Nos. PCT/US2020/035568, and PCT/US2020/035574 are hereby incorporated by reference herein in their entireties.

Extracellular vesicles (EVs) are biological particles encapsulated with a phospholipid bilayer. EVs are naturally released biological particles from cells but cannot replicate by themselves. EVs have diameter around 20 nanometers (nm) to a few microns but mostly are smaller than around 350 nm. A wide variety of EV subtypes have been proposed as defined by their size, cellular source, and function, including exosomes (˜20-150 nm), ectosomes (˜150-1000 nm) and apoptotic bodies (˜1-5 um). EVs can be found in biological fluids including blood, urine, and cerebrospinal fluid. They also release into the growth medium of cultured cells. They carry various proteins, nucleic acids, metabolites, and even organelles from their parent cells. In particular, EVs carry some biomarkers/biological molecules on their surfaces, the so-called cluster of differentiation (CD). These biomarkers, which are originated from their parent cells, have specialized functions in physiological processes and intercellular communication processes such as modulation of the immune system, inflammation reactions, and tissue regeneration. Therefore, detection of these CDs can be helpful in certain clinical applications such as early disease detection and treatment by diagnostic biomarkers and therapeutics.

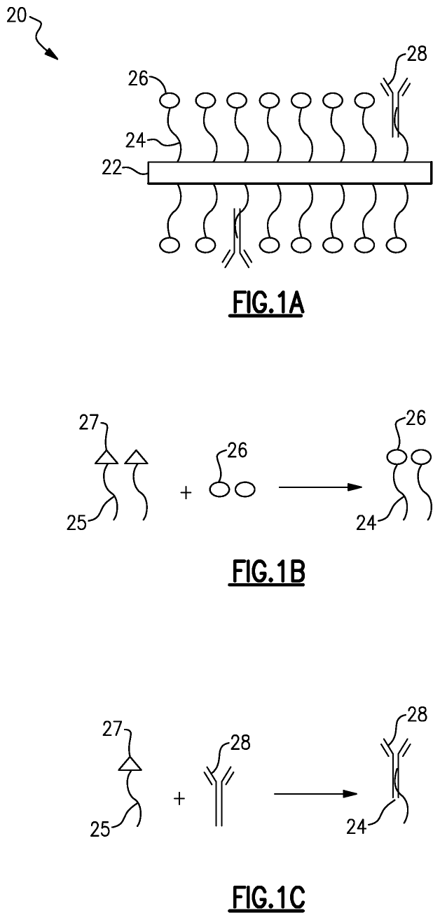

Referring now to FIG. 1A, fluorophores 20 are schematically shown. Fluorophores 20 generally comprise an inorganic nano-scale carrier 22, a linker 24, a fluorescent entity 26, as well as one or more biomolecules 28 (such as antibodies). The biomolecules 28 can be selected to interact with biomarkers on EVs. Example biomarkers include surface markers such as MHC, CD9, CD63,CD81 etc. This interaction connects the fluorophore 20 to the EV by interaction between the biomarker 28 on the fluorophore and the CD(s) so that the EV. In this way, the EV can be detected (and counted, identified, etc.) by detection of the fluorophore 20, as discussed in more detail below.

The carrier 22 is, in one example, a BNNT or CNT carrier. The carrier 22 can be fabricated by any known method.

In a particular example, the carrier 22 is a multi-walled BNNT or CNT carrier, where each BNNT or CNT has multiple co-axial shells of hexagonal boron nitride (h-BN for BNNTs) or graphene (for CNTs), with a typical external diameter of more than about 1 nm but less than about 80 nm. The length of these BNNTs and CNTs is between about 1-5000 nm. In other examples, the carrier 22 can be another nano-scale inorganic material, such as boron nitride (h-BN) nanosheets/nanoparticles and graphene/graphite nanosheets/nanoparticles. Boron nitride nanodots and carbon nanodots are also contemplated. In one example, as is more fully described in International Application No. PCT/US2020/035574, the nanodots are processed by mechanical agitation to encourage the formation of imperfections in the nanostructure of the dots, which imperfections encourage/enable bonding to linkers 24, which in turn enables more linkers 24 and thus more fluorescent entities 26 to bond to the nanodot and improve fluorescence of the resulting fluorophore 20.

Referring now to FIGS. 1B-C, the linker 24 is an amphiphilic polymeric linker. That is, the linker 24 includes a hydrophobic region 25 and a hydrophilic region 27. The hydrophobic region 25 non-covalently bonds to the nanotube carrier 22, while the hydrophilic region 27 is covalently bonded to the fluorescent entity 26 (or another entity, as will be discussed below). One example linker is DSPE-PEGₙ (1,2-distearoyl-sn-glycero-3-phosphoethanolamine-N-[(polyethylene glycol)ₙ]), where n is a number of polyethylene glycol (PEG) molecules in a PEG chain. Other linkers 24 can similarly include a PEG chain (or a different chain) which varies in length.

The hydrophilic region 27 is covalently bonded to the biomolecule 28 (such as an antibody, nucleic acid, etc). One example linker 24 is DSPE-PEGₙ (1,2-distearoyl-sn-glycero-3-phosphoethanolamine-N-[(polyethylene glycol)ₙ]), where n is a number of polyethylene glycol (PEG) molecules in a PEG chain. Other linkers 24 can similarly include a PEG chain (or a different chain) which varies in length.

In one example, as is more fully described in patent application Ser. No. 15/953,200, the liker 24 has a molecular weight of greater than about 1000 Da (which corresponds to a stretched linker length of about 5-10 nm for a linker 24 with a PEG chain) and less than about 10000 Da, which allows for improved fluorescence of the resulting fluorophores 20 as compared to prior art fluorophores. In a further example, the linker 24 molecular weight is greater than about 2400 Da and less than about 10000 Da.

In addition to the DSPE-PEG linkers 24 discussed above, many other potential linkers are known in the art. For example, a linker 24 may comprise one or more groups selected from —CH2-, —CH═, —C≡, —NH—, —N═, O—, —NH2-, —N3-, —S—, —C(O)—, —C(O)2-, —C(S)—, —S(O)—, —S(O)2-, or any combination thereof. It will be appreciated that a linker comprising more than one of the above groups will be selected such that the linker 24 is stable (e.g., not prone to degradation) and biologically appropriate; for example, a linker 24 may not include two adjacent —O— groups, which would generate an unstable peroxide linkage. The linker 24 may be a straight chain, a branched chain, or may include one or more ring systems. Non-limiting exemplary linkers include a hydrophobic area which can be fatty acids, phospholipids, sphingolipids, phosphosphingolipids [such as DSPE, 1-O-hexadecanyl-2-O-(9Z-octadecenyl)-sn-glycero-3-phospho-(1′-rac-glycerol) (ammonium salt), N-octanoyl-sphingosine-1-{succinyl[methoxy(polyethylene glycol)5000, D-erythro-sphingosyl phosphoethanolamine, 1,2-diphytanoyl-sn-glycero-3-phospho-L-serine, 3-sn-phosphatidyl-L-serine (PS), glycosylphosphatidylinositol, 1,2-dioleoyl-sn-glycero-3-phosphoethanoamine but not limited). The hydrophobic unit can be used to conjugate with water soluble polymeric chains such as PEG (or PEO polyethyleneoxide), PMO (poly methyl oxazoline), PEI (polyethyleneimine), polyvinyl alcohol, polyvinylpyrolidone, polyacrylamide, polypeptide, carbohydrate anchors. The watersoluble polymeric chains are attached to the linkers at one end, and attached to the fluorescent entity (or another moiety, as discussed below) at a second end. These hydrophobic and hydrophilic units must have reactive groups as mentioned above and such that the groups conjugate together into amphiphilic linkers.

The fluorescent entity 26 is any know fluorescent dye, including but not limited to coumarins, benzoxadiazoles, acridones, acridines, bisbenzimides, indole, benzoisoquinoline, naphthalene, anthracene, xanthene, pyrene, porphyrin, fluorescein, rhodamine, boron-dipyrromethene (BODIPY) and cyanine derivatives. Many such fluorescent dyes are commercially available. The fluorescent entity 26 is bonded to the linker 24 by any appropriate method, such as by inducing a chemical reaction between the linker 24 and fluorescent entity 26, as is known in the art.

In another example, as is more fully described in International Application No. PCT/US2020/035574, fluorophores 20 can be created by covalent functionalization of the linkers 24 onto the carrier 22. In this example, the linker 24 includes a functional group “R” that interacts with the carrier 22 and a functional group “R′” that interacts with other moieties that are attached to the carrier 22, like the fluorescent dye molecules 26 and the antibodies 28. An example functional group is a hydroxyl group, though any known functional group is contemplated. In further examples described in International Application No. PCT/US2020/035574, the carriers 22 are processed such as by mechanical agitation in polar liquid in order to form imperfections in the nanostructure of the carriers 22, which imperfections encourage/enable bonding to linkers 24 via functional groups R.

It has been verified that BNNT carriers 22 (before conjugation with linker 24 and fluorescent entity 26) alone can initiate light scattering sufficient for detection. Therefore, in another example, the BNNT carriers 22 can be used as the carriers of fluorophores 26 without linkers 24 for the detection of EVs through flow cytometry measurements.

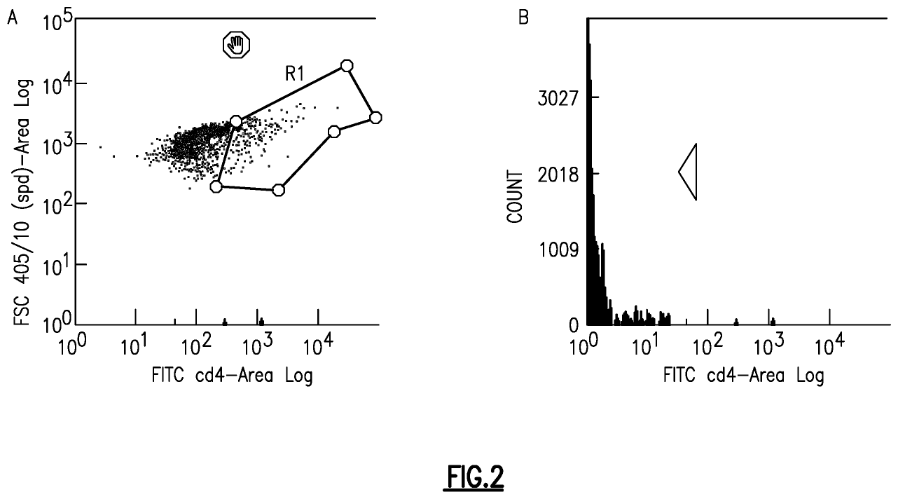

FIG. 2A shows the forward scattering (FSC) and side scattering (SSC) of laser lights of the Phosphate-buffered saline (PBS) solution that includes EVs. As shown, FSC of 10³ are initiated by laser with a wavelength of 405 nm, and lower SSC of 10² are initiated by laser with a longer wavelength of 488 nm. These levels of scattering signals are considered as “noise” for prior art methods of EV detection. Therefore, there is a detection window R1 with higher scattering strength for nano-particles, which would include EVs as well as the carriers 22 of the fluorophores 20. The flow rate for all measurements discussed herein was 0.25 μl/s and the laser powers are 100 mW for both 405 nm and 488 nm.

FIG. 2B shows the fluorescence signals collected by the FITC channel (centered around 52 nm) for the PBS solution. As shown, only noise is detected as there are no fluorophores 20 in the sample.

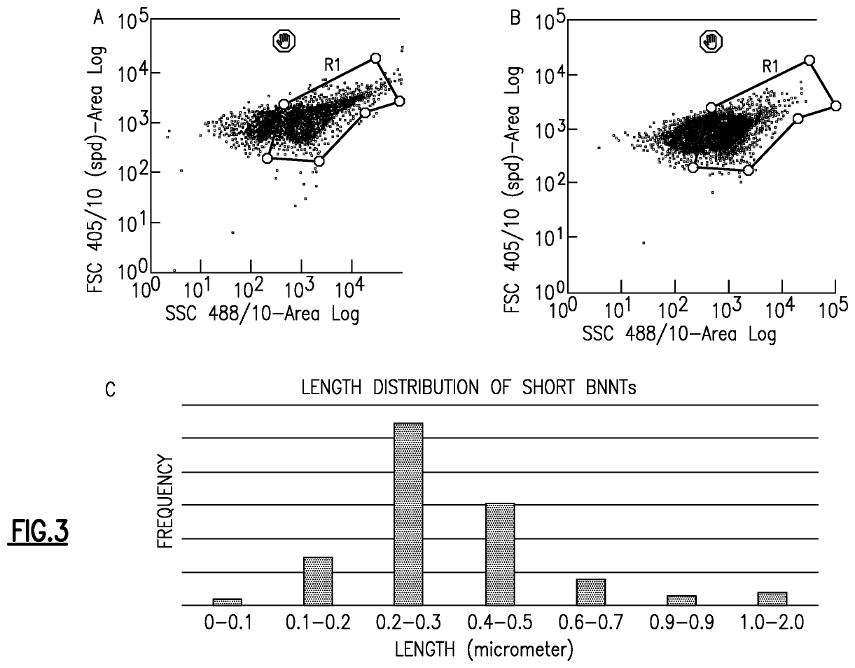

FIG. 3A shows the FSC and SSC of short BNNT carriers 22. In this example, “short” BNNTs have a length that is less than about 500 nm, with an average length of about 330 nm. The particle concentration of these BNNTs is 2.5×10⁸/ml. As shown, higher scattering signals are detected within the R1 window defined in FIG. 2A. The mean signal strength within the window is (2,698, 1,150). This means, BNNTs are detectable by laser light scattering in a flow cytometer and therefore can be quantified. This will help to quantify EVs when stained with fluorophores 20 having BNNT carriers 22 such as those described here.

FIG. 3B shows the FSC and SSC of the BNNT carriers 22 when concentrated by 4×. As shown, signals are detected within the R1 window, with the mean signal strength of (1,043, 934). This means, the detectable scattering signal depends on the concentration of the BNNTs. In this particular case, some of the strong scatterings are not being recorded at higher particle concentration (1.0×10⁹/ml). The length distribution of these short BNNTs is shown in FIG. 3C.

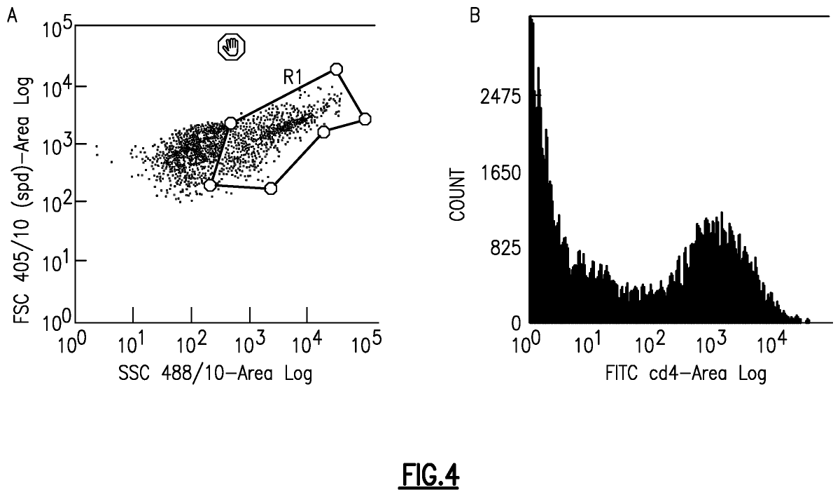

FIG. 4A shows the FSC and SSC of fluorophores 20 having short BNNT carriers, linkers 24, and a fluorescent entity 26 (in this example, FITC dye). As shown, strong scattering signals are detected within the R1 window, with a mean strength of (2,627, 1,455).

FIG. 4B shows the fluorescence signals of fluorophores 20 having short BNNT carriers, linkers 24, and a FITC entity 26. As shown, a strong signal centered around 10³ are detected. This means, both the scattering and fluorescence signal of the fluorophores 20 having short BNNT carriers, linkers 24, and a fluorescent entity 26 are also detectable under the conventional settings/circumstances for light scattering and fluorescence detection, and thus suitable for use in EV labeling and detection.

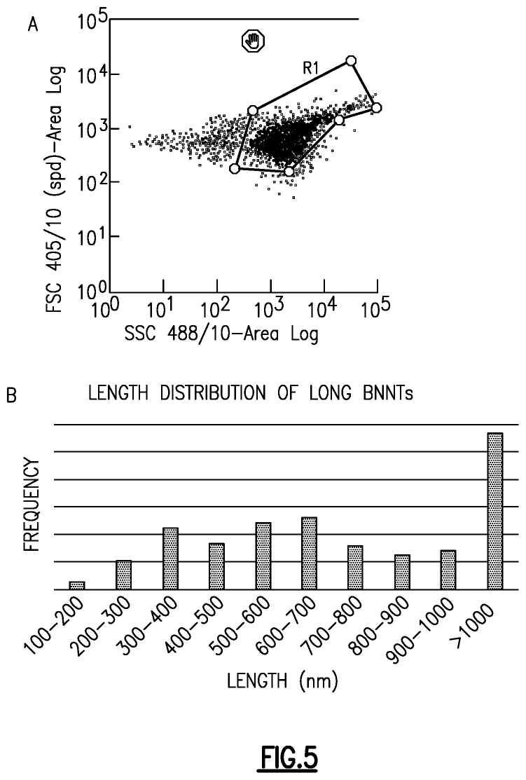

In another example, the BNNT carriers 22 can be “long” BNNTs. “long” BNNTs have a length between about 500 and 5000 nm. More particularly, long BNNTs have a length between about 500 and 2000 nm. FIG. 5B shows the length distribution of an example “long” BNNT sample, which has a mean length of 900 nm, with many BNNTs longer than 1000 nm. FIG. 5A shows the FSC and SSC of the long BNNT carriers. The particle concentration of this BNNT sample is 6×10¹⁹/ml. As shown, the strength of the scattering signals strong, with a mean strength within the R1 window of (3,221, 739).

The results discussed above show that both short and long BNNTs can initiate stronger laser scattering signals in flow cytometry, and can be used as carriers 22 for fluorophores 20 that will allow for the detection and counting of EVs.

The preceding description is exemplary rather than limiting in nature. Variations and modifications to the disclosed examples may become apparent to those skilled in the art that do not necessarily depart from the essence of this invention. The scope of legal protection given to this invention can only be determined by studying the following claims.

What is claimed is:

1. A compound, comprising:
a nanomaterial carrier;
a first linker having a first end connected to the nanomaterial carrier;
a second linker having a second end connected to the nanomaterial carrier;
a fluorescent entity connected to a second end of the first linker; and
a biomolecule connected to a second end of the second linker, wherein the biomolecule is configured to connect to a cluster of differentiation (CD) of an extracellular vesicle (EV).

2. The compound of claim 1, wherein the nanomaterial carrier is a boron nitride nanotube (BNNT) or carbon nanotube (CNT).

3. The compound of claim 1, wherein the nanomaterial is a nanodot.

4. The compound of claim 1, wherein the first end of at least one of the first and second linkers is covalently bonded to the nanomaterial carrier.

5. The compound of claim 4, wherein the first end of at least one of the first and second linkers includes a functional group, and the functional group covalently bonds the linker to the nanomaterial carrier.

6. The compound of claim 4, wherein the second end of at least one of the first and second linkers is covalently bonded to the fluorescent entity or the biomolecule via a functional group.

7. The compound of claim 1, wherein the first end of at least one of the first and second linkers is non-covalently bonded to the nanomaterial carrier.

8. The compound of claim 7, wherein at least one of the first and second linkers is amphiphilic, and includes a hydrophobic region and a hydrophilic region, and wherein the hydrophobic region is non-covalently bonded to the nanomaterial carrier.

9. The compound of claim 7, wherein the linker has a molecular weight between about 1000 and 10000 Da.

10. The compound of claim 7, wherein the nanomaterial carrier is a boron nitride nanotube.

11. The compound of claim 1, wherein at least one of the first and second linkers is DSPE-PEGₙ (1,2-distearoyl-sn-glycero-3-phosphoethanolamine-N-[(polyethylene glycol)ₙ]), where n is a number of polyethylene glycol (PEG) molecules in a PEG chain.

12. A method of detecting an extracellular vesicle, comprising:
linking at least one fluorescent entity and at least one biomolecule to a nanomaterial carrier, wherein the biomolecule is configured to connect to a cluster of differentiation (CD) of an extracellular vesicle (EV) to form a compound;
applying the compound to an EV such that the compound connects to the EV via the biomolecule to form a marked EV; and
detecting at least one of light scattering and fluorescence of the marked EV.

13. The method of claim 12, wherein the carrier is a boron nitride nanotube (BNNT) carrier, a carbon nanotube (CNT) carrier, or a nanodot.

14. The method of claim 12, wherein the linking of at least one of the fluorescent entity and the biomolecule is via a linker, and wherein the linking of the linker to the nanomaterial carrier is via a covalent bond.

15. The method of claim 14, wherein a first end of the linker includes a first functional group and a second end of the linker includes a second functional group, and wherein the first functional group covalently bonds to the nanomaterial carrier and the second functional group covalently bonds to the fluorescent entity.

16. The method of claim 12, wherein the linking of at least one of the fluorescent entity and the biomolecule is via a linker, and wherein the linking of the linker to the nanomaterial carrier is via a non-covalent bond.

17. The method of claim 16, the linker is amphiphilic, and includes a hydrophobic region and a hydrophilic region, and wherein the hydrophobic region is non-covalently bonded to the nanomaterial carrier.

18. The method of claim 16, wherein the linker has a molecular weight between about 1000 and 10000 Da.

19. The method of claim 18, wherein the nanomaterial carrier is a boron nitride nanotube.

20. The method of claim 16, wherein the linker is DSPE-PEGₙ (1,2-distearoyl-sn-glycero-3-phosphoethanolamine-N-[(polyethylene glycol)ₙ]), where n is a number of polyethylene glycol (PEG) molecules in a PEG chain.
